(19) United States
(12) Patent Application Publication
(10) Pub. No.: US 2019/0246895 A1
(43) Pub. Date: Aug. 15, 2019

(54) SYSTEM AND METHOD FOR DEVICE ASSISTED VIEWING FOR COLORBLINDNESS

(71) Applicants: Kabushiki Kaisha Toshiba, Minato-ku (JP); Toshiba TEC Kabushiki Kaisha, Shinagawa-ku (JP)

(72) Inventor: Marianne KODIMER, Huntington Beach, CA (US)

(21) Appl. No.: 15/893,365

(22) Filed: Feb. 9, 2018

(51) Int. Cl. A61B 3/06; G06T 11/00; G06T 5/00; A61B 3/00

(52) U.S. Cl. CPC A61B 3/066; G06T 11/001; G06T 5/009; A61B 3/0025; G06T 2207/20208; A61B 3/0041; G06T 2200/24; G06T 2207/10024; A61B 3/0033

(57) ABSTRACT

A system and method for adjustment of images to compensate for colorblindness includes a digital camera that generates image data corresponding to a captured color image. A processor retrieves conversion data from memory to complete a color conversion to accommodate colorblindness. The processor converts image properties associated with color in the captured color image to alternative image properties in accordance with application of the conversion data to the captured color image. The processor generates an image on the display in accordance with the alternative image data.

### TECHNICAL FIELD

This application relates generally to device assisted translation of video images. The application relates more particularly to use of a portable data device to provide user customized image translation to compensate for colorblind users.

### BACKGROUND

Vision is frequently believed to be the most important of the human senses. We can sense shapes, light levels and colors to secure an understating about our surroundings and receive information. Vision allows one to read, including books, signs, display terminals and maps. While much information can be communicated monochromatically, such as with the written text on this page, information can also be provided through color. For example, maps may be color coded to highlight locations, roads, or types of roads. Flashing red lights indicate to drivers a presence of an emergency vehicle. Flashing blue lights indicate to drivers a presence of a police vehicle.

About 1 out of 12 males and about 1 out of 20 females are color blind or color vision deficient. When one has a color vision deficiency, their perception of colors is different from what most people see. The most severe forms of these deficiencies are referred to as color blindness. People with color blindness aren't aware of differences among colors that are obvious to most people. People who don't have the more severe types of color blindness may not even be aware of their condition unless they're tested in a clinic or laboratory.

Inherited color blindness is caused by abnormal photopigments. These color-detecting molecules are located in cone-shaped cells within the retina, called cone cells. In humans, several genes are needed for the body to make photopigments, and defects in these genes can lead to color blindness.

There are three main kinds of color blindness, based on photopigment defects in the three different kinds of cones that respond to blue, green, and red light. Red-green color blindness is the most common, followed by blue-yellow color blindness. A complete absence of color vision or total color blindness is rare.

Sometimes color blindness can be caused by physical or chemical damage to the eye, the optic nerve, or parts of the brain that process color information. Color vision can also decline with age, most often because of cataracts which are a clouding and yellowing of an eye's lens.

Color blindness can significantly affect a person's condition. People with color blindness may not be able to discern differences in colors such as might be found in roadmaps or displays on computer screens. Common red-green color blindness makes it difficult or impossible to discern all colors that have some red or some green as part of the viewed color. For example, a red-green color blind person will confuse a blue and a purple because they can't differentiate the red component of the purple color. Unfortunately, many software and hardware user interfaces use color to differentiate user interface components, and further, communicate meaning such as error, warning, good, bad, danger, etc. This provides potential confusion in areas such as reading a color coded map, bus or train route, or directory.

### SUMMARY

In accordance with an example embodiment of the subject application, a system and method for adjustment of images to compensate for colorblindness includes a digital camera that generates image data corresponding to a captured color image. A processor retrieves conversion data from memory to complete a color conversion to accommodate colorblindness. The processor converts image properties associated with color in the captured color image to alternative image properties in accordance with application of the conversion data to image data of the captured color image. The processor generates an image on the display in accordance with the alternative image data.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Various embodiments will become better understood with regard to the following description, appended claims and accompanying drawings wherein:

FIG. 1 an example embodiment of a system for device assisted viewing for colorblindness;

FIG. 2 is an example embodiment of an image adjustment system;

FIG. 3 is an example embodiment of a digital data processing device;

FIG. 4 is an example embodiment of a user testing system to determine a user's particular colorblindness characteristics;

FIG. 5 is a flowchart of operations for an example embodiment of a colorblindness test;

FIG. 6 is a flowchart for an embodiment of color gamut conversion;

FIG. 7 is an example embodiment of a converted image for users who cannot discern color at all;

FIG. 8 is an example embodiment of a map colors as perceived by someone with normal color vision;

FIG. 9 is an example embodiment of how a color blind user might perceive the map of FIG. 8; and

FIG. 10 is an example embodiment of how a map image might appear to a color blind user after conversion to accommodate their colorblindness.

### DETAILED DESCRIPTION

The systems and methods disclosed herein are described in detail by way of examples and with reference to the figures. It will be appreciated that modifications to disclosed and described examples, arrangements, configurations, components, elements, apparatuses, devices methods, systems, etc. can suitably be made and may be desired for a specific application. In this disclosure, any identification of specific techniques, arrangements, etc. are either related to a specific example presented or are merely a general description of such a technique, arrangement, etc. Identifications of specific details or examples are not intended to be, and should not be, construed as mandatory or limiting unless specifically designated as such.

Graphic designers undertake visual communication and problem-solving using one or more of typography, photography and illustration. Color selection is a powerful tool for graphic design. A graphic designer may attempt to maximize usefulness of their design by selecting color combinations that avoid confusion for color blind users. However, it is difficult to design for a color deficiency because there are many different deficiency types. A color combination that addresses a need of one type of color blindness may trigger a problem when viewed by a user with a different type of color blindness.

Color is attributed to light wavelength, suitably expressed by frequency or wavelength. Visible light extends, with lower to higher wavelength, to red, orange, yellow, green, blue and violet. These are not distinct levels, but a gradual change in color corresponding to a gradual change in frequency. White light is a combination of all colors. Colors can be separated from white light by use of prism. Such separation occurs naturally in a rainbow.

Light coloration can be altered by combinations of color primaries, forming a gamut of colors. Primary colors can be additive, such as with red-yellow-blue (RYB) or red-green-blue (RGB). Additive colors actively produce light so as to, for example, project light of a certain color onto a white background. Additive primaries are used for device displays, such as a touchscreen on a smartphone or a flat screen on a notebook computer. Images are formed by an array of picture elements (pixels), each of which is formed with sub-elements for each of the primary colors. A pixel, for example, has a triad of sub-elements such as phosphors or LEDs, one for red, one for green and one for blue. Control of brightness of some or all of these sub-elements allows for control of a pixel's color.

Digital devices generate displays by pixels defined by digital values. A color may be encoded digitally, for example, in RGB. Variants included encoding values for hue, saturation and lightness (HSL) or hue, saturation and value (HSV).

Primary colors can also be subtractive, such as with cyan-magenta-yellow-black (CMYK), such as may be readily found as ink or toner colors used by printers. Subtractive primaries absorb different wavelengths from received white light. For example, red ink absorbs all wavelengths other than red supplying the red color on white paper. Differing levels of CMYK in provide for control of coloration on printed images.

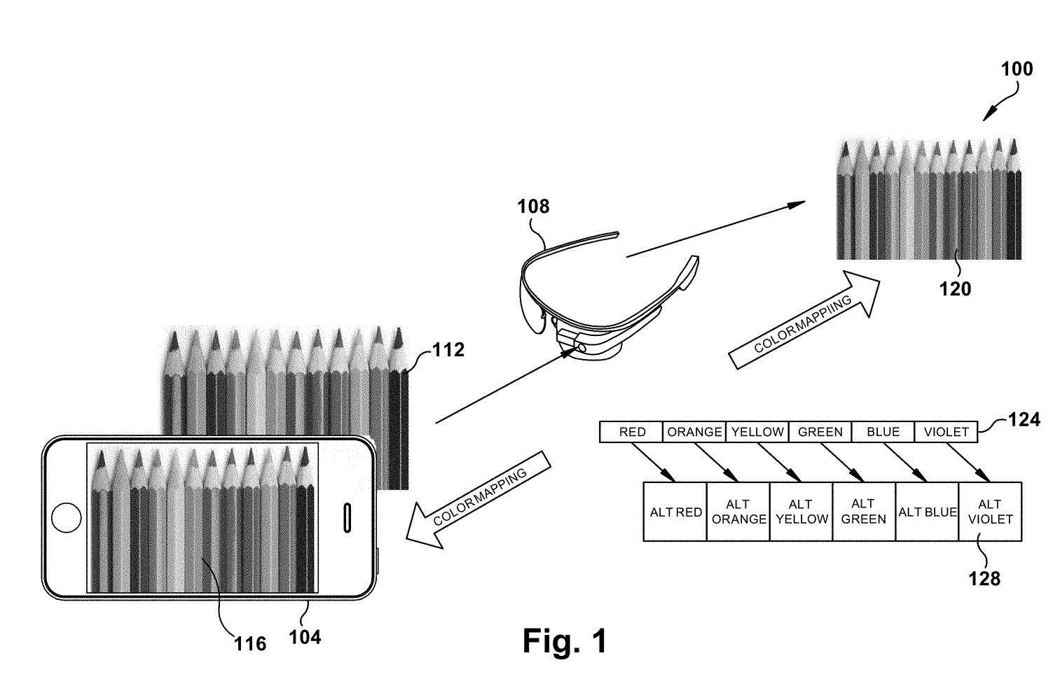

In accordance with the subject application, FIG. 1 illustrates an example embodiment of a system for device assisted viewing for colorblindness 100. In the example, portable digital devices suitably include smartphone 104 or smart glasses 108, both of which include an embedded digital camera and a display. The digital devices include software that can take a colored image captured by their camera, such as a grouping of colored pencils 112, and translate it to a color scheme that accommodates colorblindness characteristics of a device user. In the illustrated example, image adjustment is to accommodate a red-green color blindness. It will be noted that an adjusted image 116 on smartphone display or on a smart glasses viewer image have been adjusted to provide contrast to the adjusted image within the color sensing capabilities of the user. Such translation is suitably done mapping of colors from a first gamut 124 to a second gamut 128 in accordance with a user's needs. Such translation is suitably on still pictures or continuously on a video capture.

In the example embodiment of FIG. 1, as will be detailed further below, in the event a user has complete color blindness, such as only seeing in black and white or grayscale, colorization is suitably substituted with grayscale adjustments or unique patterns for each color, such as dots, dashes, lines, hatching or the like.

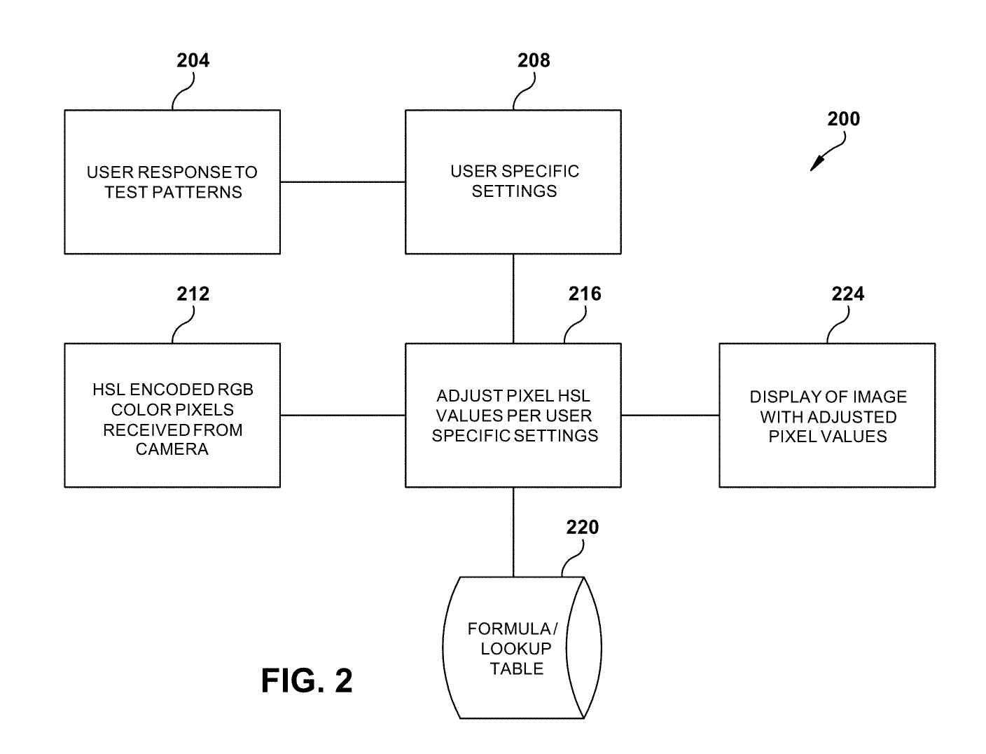

FIG. 2 illustrates an example embodiment of an image adjustment system 200 in which a formula or lookup table is suitably used for image adjustment of digitally encoded pixel value. In block 204, a user supplies their colorblindness characteristics by either inputting it directly or via a learning sequence, such as by completing a responding to one or more visual tests, such as one or more test plates used in an Ishihara colorblindness test. An example of such testing will be provided below. A user's colorblindness characteristics facilitate determination of user specific settings at block 208. Encoded pixels, such as HSL or HSV encoded pixels, are provided in camera image data at block 212. Encoded pixel values are adjusted at block 216 to accommodate the user's needs. Adjustment is suitably completed formulaically or via a lookup table stored at block 220. An adjusted image is then displayed at block 224.

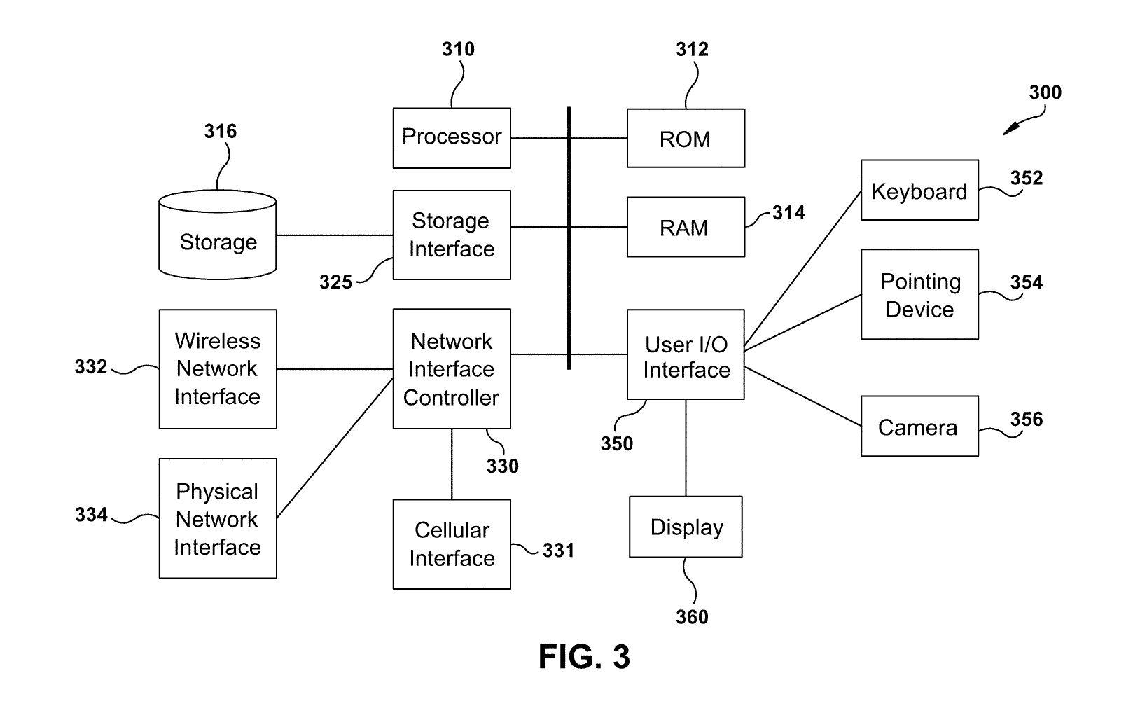

Turning now to FIG. 3, illustrated is an example embodiment of a digital data processing device 300, suitably comprising portable data devices such as a smartphone, tablet computer, notebook computer or smart glasses. Components of the data processing device 300 suitably include one or more processors, illustrated by processor 310, memory, suitably comprised of read-only memory 312 and random access memory 314, and bulk or other non-volatile storage 316, suitable connected via a storage interface 325. A network interface controller 330 suitably provides a gateway for data communication with other devices via wireless network interface 332 and physical network interface 334, as well as a cellular interface 231 such as when the digital device is a cell phone or tablet computer. A user input/output interface 350 suitably provides a gateway to devices such as keyboard 352, pointing device 354, and display 360, suitably comprised of a touch-screen display. It will be understood that the computational platform to realize the system as detailed further below is suitably implemented on any or all of devices as described above. A camera 356 is suitably included such as when the digital device is a camera or tablet computer.

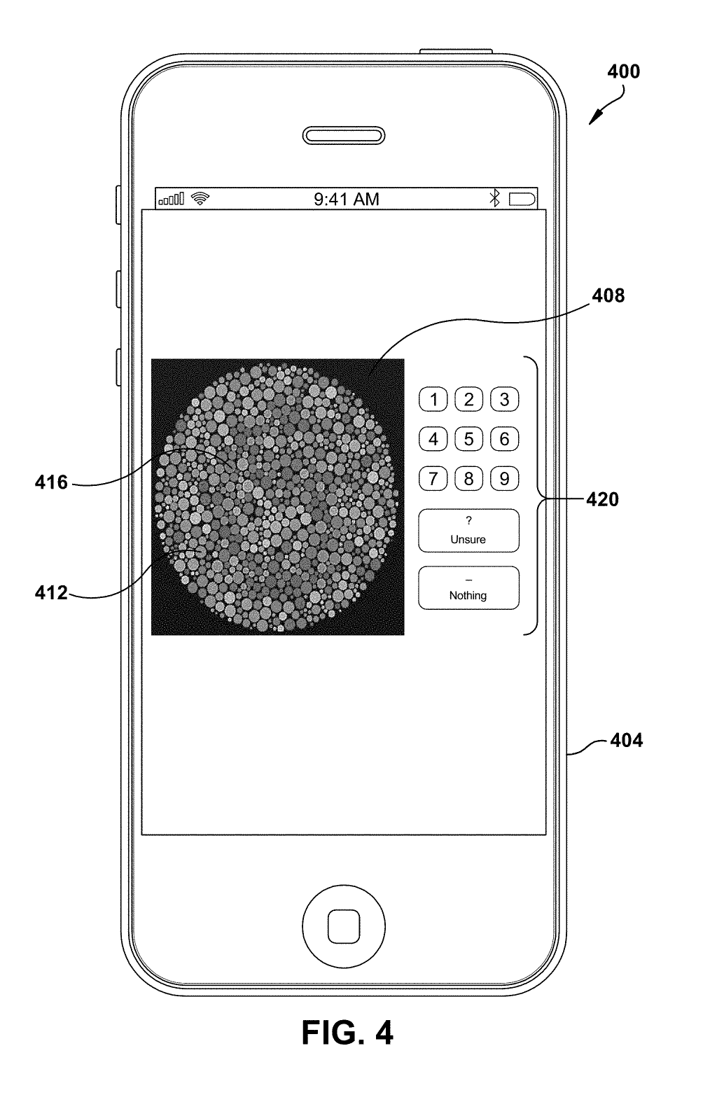

FIG. 4 is an example embodiment of a user testing system 400 to determine a user's particular colorblindness characteristics. The test is suitably run on any digital data device, advantageously on the same device that a user will be using for image transformation, such as on smartphone 404. An example vision test is provided by generating an image of an Ishihara test plate 408 on the device display. In such a test, background 412 is in a first color, while a foreground image 416 is displayed in a foreground color. In the illustrated example, a user may not be able to see the foreground image 416, a number 6, due to their particular colorblindness. User input area 420 allows for selection of a number to match the foreground character. An incorrect response or a response of “unsure” or seeing “nothing” allows for a conclusion that the user has a color sensing deficiency for the displayed color combination. A sequence of different color combination testing allows for further refinement to determine a user' colorblindness attributes.

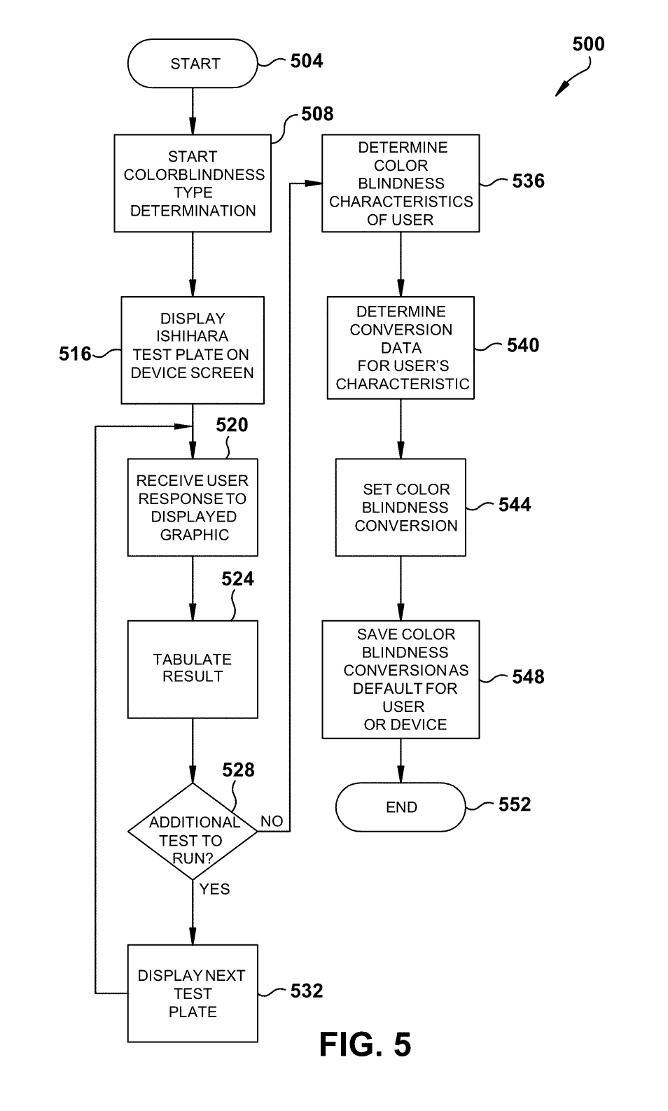

FIG. 5 is a flowchart of an example embodiment of a colorblindness test 500, such as one that might be performed with Ishihara plates as illustrated in FIG. 4. The process commences at block 504 and a test application is initiated at block 508. A first test plate is displayed at block 516 on the device screen. User input relative to the displayed plate is received at block 520 and results tabulated at block 524. A determination is made at block 528 if additional tests should be run. This determination is suitably made with a preset series of tests, or tests that are determined based on prior test results. Additional tests may be deemed unnecessary if early results provide a positive identification of a user's particular colorblindness characteristics. If more testing is to be made, a new test plate is displayed at block 532 and the process returns to block 520. If no further testing is needed, or if all tests have been completed, the user's colorblindness characteristics are determined at block 536 and appropriate conversion parameters for the user's colorblindness characteristics are determined at block 540. The conversion parameters are saved at block 544. The conversion parameters can be set as default conversion parameters on one or more user devices at block 548. The process ends at block 552.

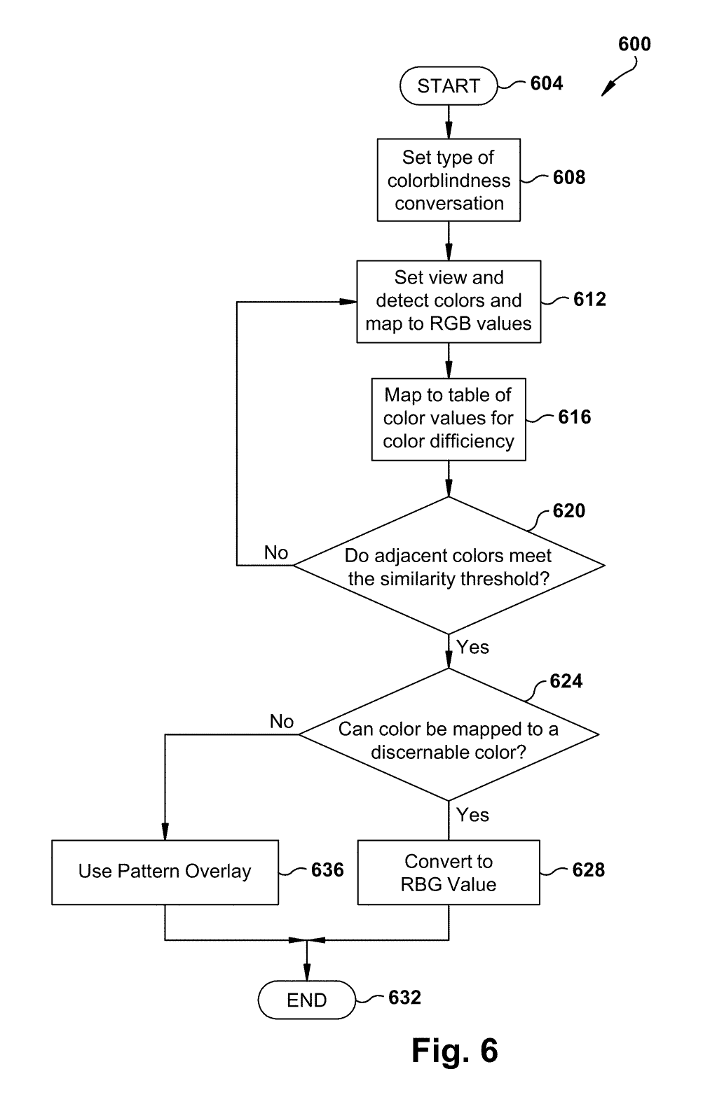

FIG. 6 illustrates a flowchart 600 of an example embodiment for color gamut conversion to enhance image contrast for colorblind users. The process commences at block 604 and a type of colorblindness conversion set at block 608. Image colors are detected at block 612 and they are mapped to appropriate colors in block 616. Adjacent colors are analyzed at block 620 to determine whether they achieve a similarity threshold. If not, the process returns to block 612 for further adjustment. If the threshold is met, a determination is made at block 624 as to whether color can be mapped to a discernable color. If so, the image can be converted to an RGB value at block 628 before the process ends at block 632. If not, a pattern overlay is implemented on the output image at block 636 before the process ends at block 632.

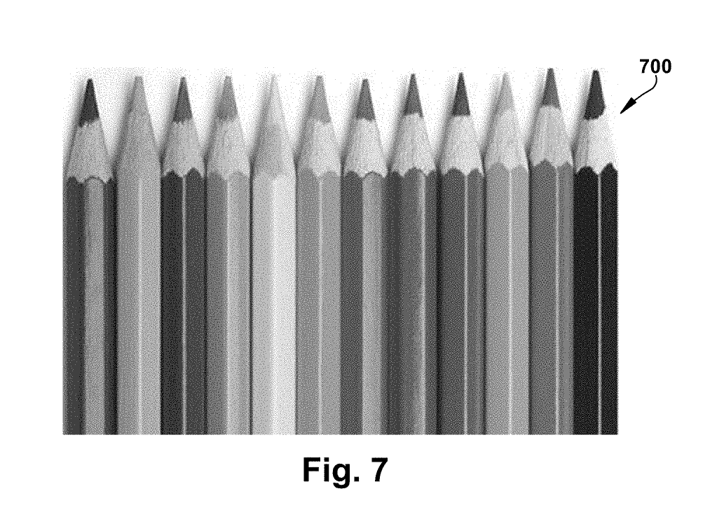

FIG. 7 is an example embodiment of a converted image for users who cannot discern color at all. Image 700 is displayed using a greyscale that provides for differentiating between pencils of different color. In an embodiment, black and white hatching can be used instead of, or in addition to, using greyscale.

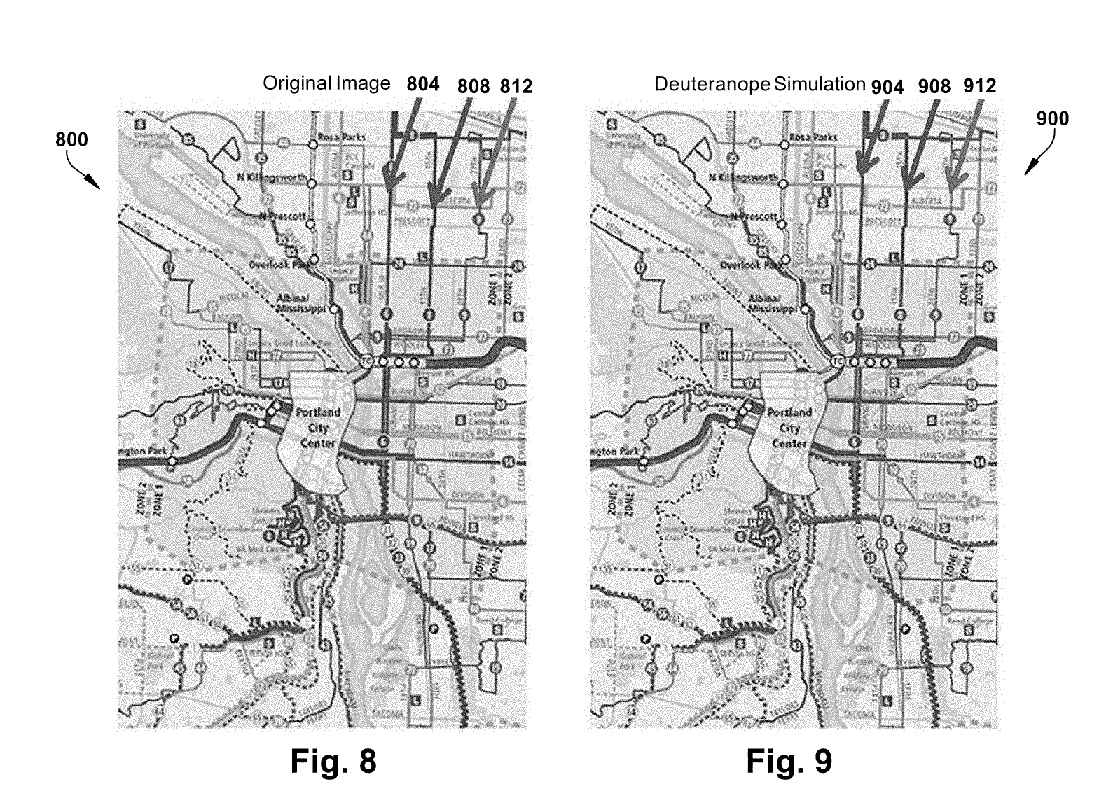

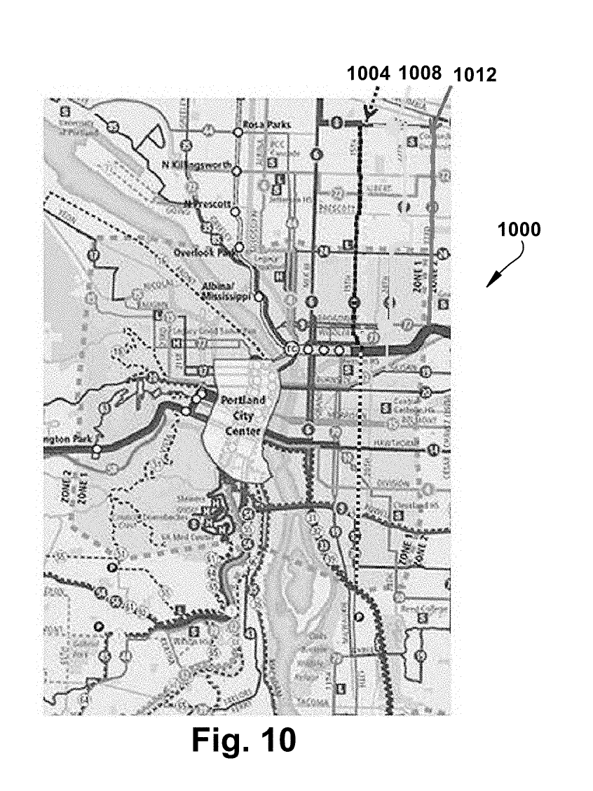

FIGS. 8-10 illustrate variations of perception on the same map image. FIG. 8 shows map colors as perceived by someone with normal color vision. It will be noted that roads illustrated in purple, such as road 804 are visually distinguishable from roads in red, such as road 808 and roads in green, such as road 812. FIG. 9 illustrates how a color blind user with a red-green deuteranomaly would perceive the same map. Note that roads 904, 908 and 912, corresponding to roads 804, 808 and 812 of FIG. 8, appear to all have the same, or nearly the same, color. FIG. 10 illustrates map 1000 wherein the image has been adjusted for the user's colorblindness. Corresponding roads 1004, 1008 and 1112 new appear visually distinct to the colorblind user. It will be noted that road 1004 employs hatching to differentiate from the others.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the spirit and scope of the inventions.

What is claimed is:

1. A system comprising:
a digital camera configured to generate image data corresponding to a captured color image;
a processor and associated memory; and
a display,
wherein the memory is configured to store conversion data corresponding to a color conversion corresponding to a color blindness type,
wherein the processor is configured to convert the image data to alternative image data in accordance with application of the conversion data to the image data, and
wherein the processor is further configured to generate an image on the display in accordance with the alternative image data.

2. The system of claim 1 wherein the processor is further configured to convert the image data to the alternative image data in accordance with a color map between encoded pixel color values in the captured color image and encoded pixel color values in the alternative image data.

3. The system of claim 2 wherein the processor is further configured to convert the image data to the alternative image data encoded to include a pattern corresponding to at least one color range.

4. The system of claim 1 wherein the processor is further configured to convert the image data to the alternative image data in accordance with values of hue, saturation, lightness or chroma, at least one of which is encoded in the captured colored image.

5. The system of claim 1 wherein the processor is further configured to select the conversion data in accordance with user input.

6. The system of claim 5 wherein the user input is comprised of a designation of the color blindness type.

7. The system of claim 5 wherein the user input is comprised of responses to at least one color blindness test pattern generated on the display by the processor.

8. A method comprising:
activating a digital camera;
generating image data corresponding to a color image captured from the camera;
retrieving conversion data corresponding to a color conversion corresponding to a color blindness type from a memory;
converting the image data to alternative image data in accordance with application of the conversion data to the image data by a processor; and
generating an image in accordance with the alternative image data on a display.

9. The method of claim 8 further comprising converting the image data to the alternative image data in accordance with a color map between encoded pixel color values in the captured color image and encoded pixel color values in the alternative image data.

10. The method of claim 9 further comprising converting the image data to the alternative image data encoded to include a pattern corresponding to at least one color range.

11. The method of claim 8 further comprising to converting the image data to the alternative image data in accordance with values of hue, saturation, lightness or chroma, at least one of which is encoded in the captured colored image.

12. The method of claim 8 further comprising selecting the conversion data in accordance with user input.

13. The method of claim 12 further comprising receiving user input comprised of a designation of the color blindness type.

14. The method of claim 12 further comprising generating at least one color blindness test pattern on the display and receiving the user input responsive thereto.

15. A portable data device comprising:
a processor and associated memory;
a user interface including a user input and a display; and
a digital camera configured to capture image data,
wherein the processor is configured to generate a prompt on the display for input relative to a color blindness type of an associated user;
wherein the input is configured to receive selection data defining the color blindness type of the associated user;
wherein the memory is configured to store conversion data corresponding to color conversion for each of a plurality of color blindness types;
wherein the processor is further configured to select conversion data in accordance with received selection data;
wherein the processor is further configured to convert the image data to alternative image data in accordance with application of the conversion data to the image data; and
wherein the processor is further configured to generate an image on the display in accordance with the alternative image data.

16. The portable data device of claim 15 wherein the selection data is comprised of a user selection corresponding to at least one of red-green color blindness, blue-yellow color blindness and complete color blindness.

17. The portable data device of claim 16 wherein the alternative image data is comprised of graphical patterns corresponding to colors represented by the captured image data.

18. The portable data device of claim 15 wherein the processor is further configured to generate the prompt comprising at least one plate of an Ishihara colorblindness test.

19. The portable data device of claim 19 wherein the processor is further configured to determine the color blindness type in accordance with the user input forming a result of completion of the Ishihara test on the at least one plate.

20. The portable data device of claim 15 wherein the processor is further configured to:
map colors encoded into captured image data to a perceived color schema based on the color blindness type;
assign values to mapped colors corresponding to a degree of similarity; and
map colors of the alternative image data to a more visually distinct color in accordance with a relative degree of similarity.
